(19) United States
(12) Patent Application Publication
(10) Pub. No.: US 2007/0167480 A1
(43) Pub. Date: Jul. 19, 2007

(54) Pure and stable tiotropium bromide

(75) Inventors: Roberta Volonte, Rovellasca (CO) (IT); Nicola Diulgheroff, Torino (IT); Francesca Scarpitta, Ivrea (TO) (IT); Alessandro Pontiroli, S. Maria della Versa (PV) (IT); Roberto Casalone, Santhia' (VC) (IT)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Sicor Inc., Irvine, CA (US)

(21) Appl. No.: 11/643,013

(22) Filed: Dec. 19, 2006

Related U.S. Application Data
(60) Provisional application No. 60/835,200, filed on Aug. 3, 2006.
(60) Provisional application No. 60/835,201, filed on Aug. 3, 2006.
(60) Provisional application No. 60/836,037, filed on Aug. 7, 2006.
(60) Provisional application No. 60/830,231, filed on Jul. 10, 2006.
(60) Provisional application No. 60/851,223, filed on Oct. 12, 2006.
(60) Provisional application No. 60/852,740, filed on Oct. 18, 2006.
(60) Provisional application No. 60/752,672, filed on Dec. 19, 2005.
(60) Provisional application No. 60/754,530, filed on Dec. 27, 2005.
(60) Provisional application No. 60/761,437, filed on Jan. 23, 2006.
(60) Provisional application No. 60/774,051, filed on Feb. 15, 2006.
(60) Provisional application No. 60/780,310, filed on Mar. 7, 2006.
(60) Provisional application No. 60/832,189, filed on Jul. 20, 2006.

(51) Int. Cl. A61K 31/44

(52) U.S. Cl. 514/291

(57) ABSTRACT

This invention relates to solvates of tiotropium bromide having a purity of at least 99%, process for preparing such pure solvates, and their use in pharmaceutical formulations. This invention also provides tiotropium bromide solvates containing less than about 0.15% area by HPLC of 2,2-dithienyl glycolic acid.

### CROSS-REFERENCE TO RELATED APPLICATIONS

This application claims the benefit of the filing date of U.S. Provisional Patent Application No. 60/836,037 filed Aug. 7, 2006; U.S. Provisional Patent Application No. 60/835,200 filed Aug. 3, 2006; U.S. Provisional Patent Application No. 60/835,201 filed Aug. 3, 2006; U.S. Provisional Patent Application No. 60/752,672 filed Dec. 19, 2005; U.S. Provisional Patent Application No. 60/754,530 filed Dec. 27, 2005; U.S. Provisional Patent Application No. 60/761,437 filed Jan. 23, 2006; U.S. Provisional Patent Application No. 60/774,051 filed on Feb. 15, 2006; U.S. Provisional. Patent Application No. 60/780,310 filed Mar. 7, 2006; U.S. Provisional Patent Application No. 60/830,231 filed Jul. 10, 2006; United States Provisional Patent Application No. 60/832,189 filed Jul. 20, 2006; U.S. Provisional Patent Application No. 60/851,223 filed Oct. 12, 2006; and U.S. Provisional Patent Application No. 60/852,740 filed Oct. 18, 2006, the disclosures of which are hereby incorporated herein by reference.

### FIELD OF THE INVENTION

The present invention is directed to pure and stable Tiotropium bromide.

### BACKGROUND OF THE INVENTION

Tiotropium bromide, (1α,2β,4β,5α,7β)-7-[(hydroxydi-2-thienylacetyl)oxy]-9,9-dimethyl-3-oxa-9-azoniatricyclo[3.3.1.0]nonane bromide or 6β,7β-epoxy-3β-hydroxy-8-methyl-1αH,5αH-tropanium bromide, di-2-thienylglycolate having the following chemical structure: 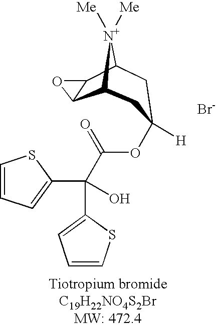
is an anticholinergic drug with specificity for muscarinic receptors. As a bronchodilator it provides therapeutic benefit in the treatment of asthma or chronic obstructive pulmonary disease (COPD). This pharmaceutical ingredient is administered by inhalation, and is available commercially as SPIRIVA® HandiHaler®.

The preparation and crystallization of Tiotropium bromide from a mixture of acetone and methanol are disclosed in U.S. Pat. No. 5,610,163.

It is reported in “Summary Basis of Approval” of the FDA (NDA 21-395) that Tiotropium bromide is subject to non-enzymatic hydrolysis to N-methyl scopine and 2,2-dithienyl glycolic acid, as illustrated by the following scheme.

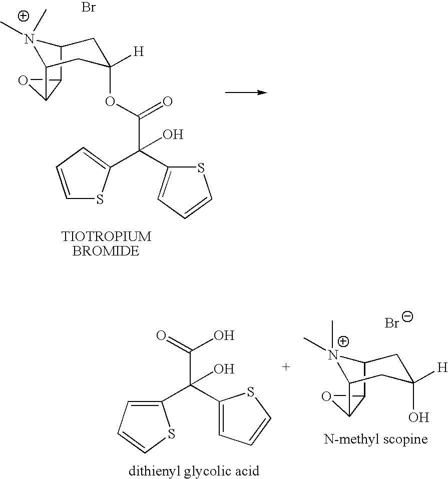

Degradation of the Tiotropium bromide API in capsules which were removed from their protective package and exposed to air and humidity were reported in the early chemistry review of NDA 21-395. The level of one degradant was reported to be lower than 1.0% (HPLC).

As a result, marketed tiotropium bromide is packed in a moisture-resistant foil blister, and it is recommended to remove the capsule from the package only immediately before use, as exposure to moisture in the air can cause decomposition.

Like any synthetic compound, Tiotropium bromide can contain extraneous compounds or impurities that can come from many sources. Some of these extraneous compounds or impurities may be unreacted starting materials, by-products of the reaction including the products of side reactions, or degradation products; wherein the degradation products are related to the stability of the API during storage. Impurities in Tiotropium bromide or any active pharmaceutical ingredient (API) are undesirable and, in extreme cases, might even be harmful to a patient being treated with a dosage form containing the API.

Therefore, there is a need in the art for stable Tiotropium bromide, and also for pure Tiotropium bromide solvates.

### SUMMARY OF INVENTION

In accordance with one aspect, the present invention provides a Tiotropium bromide solvate of the following formulas: 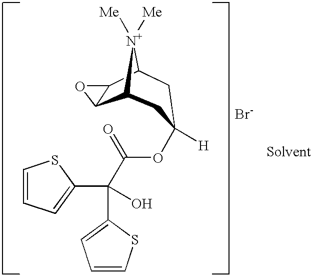
having a purity of at least 99% area by HPLC.

In another aspect, the present invention provides a Tiotropium bromide solvate, with less than about 0.15% area by HPLC of 2,2-dithienyl glycolic acid of the following formula.

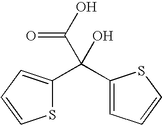

In yet another aspect, the present invention provides Tiotropium bromide solvate with a purity of at least 99% area as measured by HPLC, and with less than about 0.15% area as measured by HPLC of 2,2-dithienyl glycolic acid.

In one aspect, the present invention provides stable Tiotropium bromide solvate.

In another aspect, the present invention provides stable Tiotropium bromide solvate with a purity of at least 99% area as measured by HPLC, and with less than about 0.15% area by HPLC of 2,2-dithienyl glycolic acid.

In yet another aspect, the present invention provides an HPLC method for determining the purity of Tiotropium bromide solvate, and the amount of 2,2-dithienyl glycolic acid in Tiotropium bromide. The method comprises:
(a) combining Tiotropium bromide sample with a mixture of acetonitrile:acetic acid in water in a ratio of about 0.1%:99.9%, to obtain a solution;
(b) injecting the solution into a 250×4.6 mm×0.5 μm CPS Hypersil (or similar) column;
(c) eluting the sample from the column at about 3.63 min using a mixture of perchloric acid:water in a ratio of 7:3 (referred to as eluent A) and acetonitrile (referred to as eluent B) as an eluent; and
(d) measuring the 2,2-dithienyl glycolic acid content in the relevant sample with a UV detector.

In one aspect, the present invention provides a process for preparing stable Tiotropium bromide solvate having a purity of at least 99% area by HPLC and with less than about 0.15% area of 2,2-dithienyl glycolic acid as measured by HPLC, comprises crystallizing Tiotropium bromide from a solvent system comprising an organic acid wherein the ratio of Tiotropium bromide to the solvent system is of at least about 1 to about 5, respectively. Organic acids which may be utilized as part of the present invention include, but are not limited to, acetic acid, propanoic acid, oxalic acid, maleic acid, fumaric acid, and tartaric acid. In a preferred embodiment, the organic acid is acetic acid.

In another aspect, the present invention provides process for preparing stable Tiotropium bromide solvate having a purity of at least 99% area by HPLC and with less than about 0.05% area of 2,2-dithienyl glycolic acid as measured by HPLC, comprises crystallizing Tiotropium bromide from a solvent system comprising an organic acid wherein the ratio of Tiotropium bromide to the solvent system is of at least about 1 to about 10, respectively. Organic acids which may be utilized as part of the present invention include, but are not limited to, acetic acid, propanoic acid, oxalic acid, maleic acid, fumaric acid, and tartaric acid. In a preferred embodiment, the organic acid is acetic acid.

In yet another aspect, the present invention provides a process for preparing stable Tiotropium bromide solvate having a purity of at least 99% area by HPLC and with less than about 0.02% area of 2,2-dithienyl glycolic acid as measured by HPLC, comprises crystallizing Tiotropium bromide from a solvent system comprising an organic acid wherein the ratio of Tiotropium bromide to the solvent system is of at least about 1 to about 20, respectively. Organic acids which may be utilized as part of the present invention include, but are not limited to, acetic acid, propanoic acid, oxalic acid, maleic acid, fumaric acid, and tartaric acid. In a preferred embodiment, the organic acid is acetic acid.

In one aspect, the present invention provides a process for preparing stable Tiotropium bromide solvate having a purity of at least 99% area by HPLC and with less than about 0.01% area of 2,2-dithienyl glycolic acid as measured by HPLC, comprises crystallizing Tiotropium bromide from a solvent system comprising an organic acid wherein the ratio of Tiotropium bromide to the solvent system is of at least about 1 to about 30, respectively. Organic acids which may be utilized as part of the present invention include, but are not limited to, acetic acid, propanoic acid, oxalic acid, maleic acid, fumaric acid, and tartaric acid. In a preferred embodiment, the organic acid is acetic acid.

In another aspect, the present invention provides a pharmaceutical composition comprising stable Tiotropium bromide solvate with a purity of at least 99% area as measured by HPLC, and with less than about 0.15% area as measured by HPLC of 2,2-dithienyl glycolic acid, and pharmaceutically acceptable excipients.

In yet another aspect, the present invention provides a process for preparing pharmaceutical composition comprising stable Tiotropium bromide solvate with a purity of at least 99% area as measured by HPLC, and with less than about 0.15% area as measured by HPLC of 2,2-dithienyl glycolic acid, and pharmaceutically acceptable excipients.

In one aspect, the present invention provides the use of the stable Tiotropium bromide solvate with a purity of at least 99% area as measured by HPLC, and with less than about 0.15% area by HPLC of 2,2-dithienyl glycolic acid, of the present invention for the manufacture of a pharmaceutical composition.

In yet another embodiment, the present invention encompasses a process for preparing Tiotropium bromide with less than about 0.15% area by HPLC of 2,2-dithienyl glycolic acid comprising the steps of
(a) obtaining one or more samples of one or more Tiotrpium bromide batches;
(b) measuring the level of 2,2-dithienyl glycolic acid in each of the samples of (a);
(c) selecting the Tiotropium bromide batch that comprises a level of 2,2-dithienyl glycolic acid of less than about 0.15% area by HPLC, based on the measurement or measurements conducted in step (b); and
(d) using the batch selected in step (c) to prepare said any Tiotropium bromide comprising less than about 0.15% area by HPLC of 2,2-dithienyl glycolic acid.

### DETAILED DESCRIPTION OF THE INVENTION

An analysis of Spiriva™ capsules, containing monohydrate tiotropium bromide, supports the hydrolysis theory of Tiotropium bromide degradation by demonstrating that the level of dithienyl glycolic acid in Tiotropium bromide monohydrate is above 0.7% area by HPLC. In contrast, the present invention succeeds to provide substantially pure Tiotropium bromide solvate. The present invention also provides stable Tiotropium bromide solvate i.e., Tiotropium bromide solvate that is significantly less prone to hydrolysis upon storage. According to general ICH guidelines, acceptable purities of drug products for human treatment are generally greater than 99.0% area by HPLC, and preferably greater than 99.55% area as measured by HPLC, where the content of each single impurity is preferably less than about 0.15% area as measured by HPLC. Thus, providing such pure Tiotropium bromide solvate, which is also less prone to decomposition upon storage, is preferable considering that prolonged shelf life is an advantage for industrial manufacturing and safety of patients.

The present invention provides Tiotropium bromide solvate of the following formula: 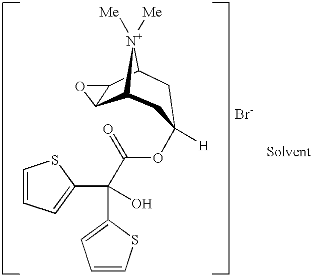
having a purity of at least 99% area by HPLC. Preferably, the above Tiotropium bromide has purity ranging from about 99% area to about 100% area as measured by HPLC, more preferably ranging from about 99.5% area to about 100% area as measured by HPLC, and most preferably ranging from about 99.7% area to about 100% area as measured by HPLC. One skilled in the art would recognize that the solvate may contain any number of solvent molecules.

Typically, the term “solvate” as used herein, refers to a substance that includes any solvent other than water at levels of more than 1%. Preferably, the solvate form of Tiotropium bromide is selected from a group consisting of an alcoholate and an acetic acid solvate. Preferably, the alcoholate is a C₁₋₈ alcoholate, more preferably a C₁₋₆ alcoholate, even more preferably a C₁₋₅ alcoholate, and most preferably a C₁₋₄ alcoholate. Preferably, the C₁₋₄ alcoholate is selected from the group consisting of methanolate, ethanolate, isopropanolate, n-propanolate and n-butanolate. Most preferably, the C₁₋₄ alcoholate is methanolate, ethanolate or n-propanolate.

The present invention also provides Tiotropium bromide solvate containing less than about 0.15% area of 2,2-dithienyl glycolic acid as measured by HPLC. Preferably, the said Tiotropium bromide is with about 0.15% area by HPLC to the detection limit of an HPLC method of 2,2-dithienyl glycolic acid.

The terms “detection limit” or “detection limit of an HPLC method” refer to any HPLC method used to determine the purity of Tiotropium bromide, and in particular, to determine the amount of 2,2-dithienyl glycolic acid in any Tiotropium bromide sample. Preferably, the detection limit is the detection limit of the HPLC method used in the present invention, or of any other equivalent method.

Preferably, Tiotropium bromide solvate is containing less than about 0.05% area as measured by HPLC of 2,2-dithienyl glycolic acid, more preferably, the said Tiotropium bromide is with about 0.05% area by HPLC to the detection limit of an HPLC method of 2,2-dithienyl glycolic acid.

Preferably, Tiotropium bromide solvate is containing less than about 0.02% area as measured by HPLC of 2,2-dithienyl glycolic acid, more preferably, the said Tiotropium bromide is with about 0.02% area by HPLC to the detection limit of an HPLC method of 2,2-dithienyl glycolic acid.

Preferably, Tiotropium bromide solvate is containing less than about 0.01% area as measured by HPLC of 2,2-dithienyl glycolic acid, more preferably, the said Tiotropium bromide is with about 0.01% area by HPLC to the detection limit of an HPLC method, of 2,2-dithienyl glycolic acid.

The present invention further provides Tiotropium bromide solvate with a purity of at least 99% area as measured by HPLC, and containing less than about 0.15% area as measured by HPLC of 2,2-dithienyl glycolic acid.

The present invention also provides stable Tiotropium bromide solvate. As used herein, the term “stable,” in reference to Tiotropium bromide, means Tiotropium bromide wherein the level of a specific impurity does not increase to more than a specific limit, when maintained at a specific relative humidity and temperature for a specific period of time. More specifically, the term “stable” means Tiotropium bromide wherein the level of the 2,2-dithienyl glycolic acid, shown below, does not increase to more than 0.15% of the total amount of tiotropium bromide area as measured by HPLC, when maintained at a temperature ranging from about 4° C. to about 30° C., for at least about two months.

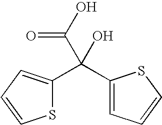

The present invention provides stable Tiotropium bromide solvate with a purity of at least 99% area as measured by HPLC, and containing less than about 0.15% area as measured by HPLC of 2,2-dithienyl glycolic acid.

The stability and purity of Tiotropium bromide were tested. Data indicated that when storing Tiotropium bromide monohydrate, at a temperature above 4° C., an increase in the content of the 2,2-dithienyl glycolic acid was detected. However, when Tiotropium bromide solvate, such as hemi-ethanolate, was stored at a temperature above 4° C., the presence of 2,2-dithienyl glycolic acid was detected only after two months, and even then, the level was significantly lower than the level detected in the monohydrate product.

The purity of Tiotropium bromide, as well as the amount of 2,2-dithienyl glycolic acid in Tiotropium bromide is determined by an HPLC method comprising:
(a) combining Tiotropium bromide sample with a mixture of acetonitrile:acetic acid in water in a ratio of about 0.1%:99.9%, to obtain a solution;
(b) injecting the solution into a 250×4.6 mm×0.5 μm CPS Hypersil (or similar) column;
(c) eluting the sample from the column at about 3.63 min using an eluent mixture of perchloric acid:water in a ratio of 7:3 (referred to as eluent A) and acetonitrile (referred to as eluent B) as an eluent; and
(d) measuring the 2,2-dithienyl glycolic acid content in the relevant sample with a UV detector.

The eluent used may be a mixture of eluent A and eluent B, wherein the ratio of them varies over the time, i.e. a gradient eluent. At the time 0 minutes, the eluent contains 70% of eluent A and 300% of eluent B. At 23 minutes, the eluent contains 55% of eluent A and 45% of eluent B. At 30 minutes, the eluent contains 50% of eluent A and 50% of eluent B. At 35 minutes, the eluent contains 50% of eluent A and 50% of eluent B. At 40 minutes, the eluent contains 35% of eluent A and 65% of eluent B, and at 41 minutes, the eluent contains 70% of eluent A and 30% of eluent B.

Preferably, the 2,2-dithienyl glycolic acid content is measured at a wave-length of 240 nm.

Such pure and stable Tiotropium bromide solvates can be prepared by a process comprises crystallizing Tiotropium bromide from a suitable solvent system; wherein the solvent system comprises acetic acid.

The process for preparing stable Tiotropium bromide solvate having a purity of at least 99% area by HPLC and with less than about 0.15% area of 2,2-dithienyl glycolic acid as measured by HPLC, comprises crystallizing Tiotropium bromide from a solvent system comprising acetic acid; wherein the ratio of Tiotropium bromide to the solvent system is of at least about 1 to about 5, respectively.

The process for preparing stable Tiotropium bromide solvate having a purity of at least 99% area by HPLC and with less than about 0.05% area of 2,2-dithienyl glycolic acid as measured by HPLC, comprises crystallizing Tiotropium bromide from a solvent system comprising an organic acid wherein the ratio of Tiotropium bromide to the solvent system is of at least about 1 to about 10, respectively. Organic acids which may be utilized as part of the present invention include, but are not limited to, acetic acid, propanoic acid, oxalic acid, maleic acid, fumaric acid, and tartaric acid. In a preferred embodiment, the organic acid is acetic acid.

The process for preparing stable Tiotropium bromide solvate having a purity of at least 99% area by HPLC and with less than about 0.02% area of 2,2-dithienyl glycolic acid as measured by HPLC, comprises crystallizing Tiotropium bromide from a solvent system comprising an organic acid wherein the ratio of Tiotropium bromide to the solvent system is of at least about 1 to about 20, respectively. Organic acids which may be utilized as part of the present invention include, but are not limited to, acetic acid, propanoic acid, oxalic acid, maleic acid, fumaric acid, and tartaric acid. In a preferred embodiment, the organic acid is acetic acid.

The process for preparing stable Tiotropium bromide solvate having a purity of at least 99% area by HPLC and with less than about 0.01% area of 2,2-dithienyl glycolic acid as measured by HPLC, comprises crystallizing Tiotropium bromide from a solvent system comprising an organic acid wherein the ratio of Tiotropium bromide to the solvent system is of at least about 1 to about 30, respectively. Organic acids which may be utilized as part of the present invention include, but are not limited to, acetic acid, propanoic acid, oxalic acid, maleic acid, fumaric acid, and tartaric acid. In a preferred embodiment, the organic acid is acetic acid.

Preferably, the crystallization process comprises providing a solution of tiotropium bromide in the solvent system comprising organic acid, and cooling to obtain a suspension. Preferably, the organic acid is acetic acid.

In yet another embodiment, the present invention encompasses a process for preparing Tiotropium bromide with less than about 0.15% area by HPLC of 2,2-dithienyl glycolic acid comprising the steps of
(a) obtaining one or more samples of one or more Tiotrpium bromide batches;
(b) measuring the level of 2,2-dithienyl glycolic acid in each of the samples of (a);
(c) selecting the Tiotropium bromide batch that comprises a level of 2,2-dithienyl glycolic acid of less than about 0.15% area by HPLC, based on the measurement or measurements conducted in step (b); and
(d) using the batch selected in step (c) to prepare said any Tiotropium bromide comprising less than about 0.15% area by HPLC of 2,2-dithienyl glycolic acid.

Typically, the Tiotropium bromide of step (a) comprises a sufficiently low level of 2,2-dithienyl glycolic acid. More preferably, the Tiotropium bromide of step (a) comprises less than about 0.15% area by HPLC of 2,2-dithienyl glycolic acid.

When the sample of Tiotropium bromide of formula II of step (a) comprises more than about 0.15% area by HPLC of 2,2-dithienyl glycolic acid, according to the measurement in step (b), the sample may be purified, prior to performing step (c).

Typically, the purifed Tiotropium bromide comprises a lower level of 2,2-dithienyl glycolic acid than the level present before purification. Preferably, the tiotrpium bromide sample of step (a) obtained after purification, comprises less than about 0.15% area by HPLC of 2,2-dithienyl glycolic acid.

Unless specified otherwise, the tiotropium bromide of step (d) of the above process may be any form tiotropium bromide, including, for example, crystalline forms and amorphous form of tiotropium bromide.

The Tiotropium bromide used as a starting material for the crystallization processes may be prepared, for example, according to the process disclosed in Co-pending application No. 60/835,201 entitled PROCESS FOR THE PREPARATION OF TRIOTROPIUM BROMIDE filed in the U.S. Patent and Trademark Office on Aug. 3, 2006, according to the process disclosed in Co-pending application No. 60/830,231 entitled PROCESS FOR THE PREPARATION OF TIOTROPIUM BROMIDE filed in the U.S. Patent and Trademark Office on Jul. 10, 2006, or by any other process known to one skilled in the art.

The process disclosed in Co-pending application Nos. 60/830,231 and 60/835,201 disclose combining methyl-di-(2-thienyl)-glycolate of formula I, an inorganic base, a polar organic solvent, and scopine salt of formula II-s, 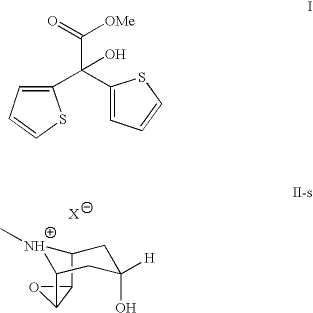
containing about 0.5% to about 40% of salts; heating; recovering; and adding an organic solvent.

The process may be performed according to the following scheme:

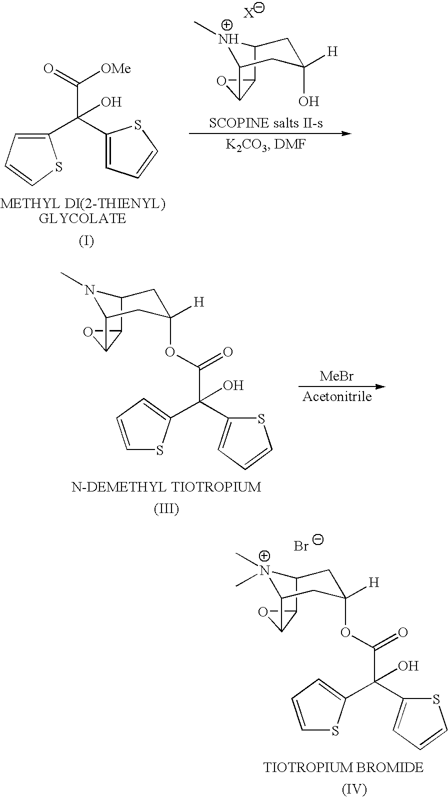

The glycolate of formula I may be prepared by combining 2-bromo-thiophene of the following formula, 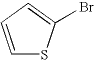
Mg, and an ethereal solvent; combining with dimethyloxalate of the following formula, and quenching.

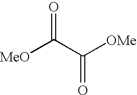

Combining 2-bromo-tiophene, Mg, and an ethereal solvent provides a Grignard reagent that can be prepared, for example, according to the process disclosed in Nyberg, K. Acta Chemica Scandinavica, 24, 1970, 1590-1596.

Methyl di-(2-thienyl)glycolate of formula I may be purified by crystallization from a mixture of ethanol and heptane, absolute ethanol and heptane, isopropanol and heptane, or from toluene and heptane.

Preferably, the scopine salt of formula II-s is suspended in a polar organic solvent. Preferably, the polar organic solvent is selected from a group consisting of dimethylformamide, N-methyl-2-pyrrolidone, dimethylacetamide, dimethylsulfoxide, acetonitrile, sulfolane, and mixtures thereof. More preferably, the polar organic solvent is dimethylformamide Preferably, the salt is an HBr salt.

Preferably, the inorganic base and methyl-di-(2-thienyl)-glycolate of formula I are added to the suspension. More preferably, the inorganic base is anhydrous. Even more preferably, the inorganic base has a pKa of about 6 to about 12, more preferably of about 9 to about 10. Yet even more preferably, the inorganic base is selected from a group consisting of: K₂CO₃, NaHCO₃, Na₂CO₃, Li₂CO₃, Cs₂CO₃, KO^{t}Bu, and LiO^{t}Bu. Most preferably, the inorganic base is K₂CO₃. The inorganic base is added in an amount of 0.45 to 2.5 mole equivalent per mole equivalent, more preferably, 2 to 2.5 mole equivalent per mole equivalent of scopine salt.

Preferably, methyl-di-(2-thienyl)-glycolate of formula I is added in the form of a solution in the polar organic solvent. Preferably, the inorganic base and methyl-di-(2-thienyl)-glycolate of formula I are added at a temperature of about 25° C. to about 65° C., more preferably at about 60° C. to about 65° C.

Preferably, the suspension containing all the above substances is heated to a temperature of below 70° C., more preferably to a temperature ranging from about 25° C. to about 65° C., even more preferably at a temperature ranging from about 60° C. to about 65° C., and most preferably at a temperature ranging from about 63° C. to about 65° C. Preferably, heating is done under reduced pressure. Preferably, the pressure is of about 70 to about 100 milibar. Preferably, nitrogen is bubbled during the reaction, through a second inlet. More preferably, nitrogen is bubbled in a rate of about 1.8 to about 2.6 L/min, even more preferably of about 2.0 to about 2.4 L/min, and yet even more preferably of about 2.2 to about 2.4 L/min. Heating under pressure, while bubbling nitrogen from a second inlet, assists in evaporating methanol, which is formed during the reaction. As such, the reaction shifts towards the formation of the product. Preferably, heating is done for a time ranging from about 17 to about 24 hours, more preferably for about 18 to about 20 hours.

N-demethyl-tiotropium of formula III may be recovered by a) cooling the suspension; b) adding an acid; c) extracting the aqueous phase; d) adding a base to the aqueous phase; e) filtering; and f) washing and drying. Preferably, the acid is HBr. Preferably, the suspension is cooled to a temperature of about 10° C. to about −10° C., more preferably to about 5° C. to about 0° C. Preferably, the addition of the acid provides a pH of about 3. Preferably, the aqueous phase is extracted with toluene. Preferably, the base is added at a temperature of about 0° C. to about 5° C. More preferably, the base is K₂CO₃ Preferably, the addition of the base causes precipitation of N-demethyl-tiotropium of formula III. Preferably, the precipitate is washed with water to obtain a pH of about 7.

Optionally, scopine base may be used. When scopine base is used, preferably a smaller amount of the inorganic base is used. Preferably, about 1 to 1.5 mole equivalent of inorganic base per mole equivalent of scopine base may be used.

After N-demethyl-tiotropium of formula III is obtained, it is converted to Tiotropium bromide by reacting with methylbromide in an organic solvent. Preferably, the organic solvent is selected from a group consisting of: C₂₋₄ nitrile, C₄₋₈ linear or cyclic ether, mixtures of C₂₋₄ nitrile and C₄₋₈ linear or cyclic ether, mixtures of C₇₋₈ aromatic hydrocarbon and C₂₋₄ nitrile, and mixtures of C₂₋₄ nitrile and C₃₋₁₀ ketone. Preferably, the C₂₋₄ nitrile is acetonitrile. A preferred C₄₋₈ linear or cyclic ether is tetrahydrofuran. Preferably, a mixture of C₂₋₄ nitrile and C₄₋₈ linear or cyclic ether is that of acetonitrile and tetrahydrofuran. A preferred mixture of C₇₋₈ aromatic hydrocarbon and C₂₋₄ nitrile is that of toluene and acetonitrile. Preferably, a mixture of C₂₋₄ nitrile and C₃₋₁₀ ketone is that of acetone and acetonitrile, and heating is conducted to a temperature of about 20° C. to about 40° C. Preferably, the solvent is acetonitrile. Preferably, heating is done to a temperature of about 20° C. to about 25° C. More, preferably, heating is done for about 12 to about 64 hours, even more preferably, for about 18 to about 22 hours.

Initially, crude Tiotropium bromide is dissolved in the solvent system which is comprised of an organic acid. Examples of such organic acids include, but are not limited to, trifluoroacetic acid, tartaric acid, maleic acid, propionic acid, oxalic acid, p-toluen sulphonic acid, methan sulphonic acid, HCl, HBr, H₂SO₄ and acetic acid. Preferably the organic acid is acetic acid.

Preferably, the solvent system comprises acetic acid, C₁₋₈ alcohol and acetic acid, C₁₋₈ alcohol, acetic acid and acetone or C₁₋₈ alcohol, acetic acid and water. Preferably, the alcoholate is a C₁₋₈ alcohol, more preferably a C₁₋₆ alcohol, even more preferably a C₁₋₅ alcohol, and most preferably a C₁₋₄ alcohol. Preferably, the C₁₋₄ alcohol is selected from the group consisting of methanolate, ethanolate, isopropanolate, n-propanolate and n-butanolate. Most preferably, the C₁₋₄ alcoholate is methanolate, ethanolate or n-propanolate.

Typically, the dissolution is achieved by heating the combination of Tiotropium bromide and the solvent system. Preferably, the heating is to a temperature ranging from about 60° C. to about 78° C., more preferably from to about 65° C. to about 78° C., most preferably from about 65° C. to about 75° C.

Preferably, the solution is then cooled to a temperature ranging from about 25° C. to about 0° C., more preferably from about 25° C. to about 5° C., and most preferably from about 5° C. to about 0° C., to induce precipitation of the crystallized product. Preferably, cooling is done over a period of about 4 to about 10 hours, more preferably from about 6 to about 9 hours, most preferably of about 8 to about 9 hours.

Typically, the suspension is maintained to increase the yield of the precipitated crystallized product. Preferably, the suspension is maintained for a time period ranging from at least about 3 hours to about 21 hours, more preferably from about 6 hours to about 12 hours, and most preferably from about 13 hours to about 18 hours.

The crystallization process may further comprise a recovery step. The precipitate may be recovered by any method known to a skilled artisan. Preferably, the recover comprises filtering the suspension, washing the filtered product, and drying.

The present invention also provides a pharmaceutical composition comprising a stable Tiotropium bromide solvate having a purity of at least 99% area as measured by HPLC, and with less than about 0.15% area by HPLC of 2,2-dithienyl glycolic acid, and pharmaceutically acceptable excipients. The present invention also provides a pharmaceutical composition comprising a stable Tiotropium bromide solvate having a purity of at least 99.3% area as measured by HPLC, and with less than about 0.15% area by HPLC of 2,2-dithienyl glycolic acid, and pharmaceutically acceptable excipients. The present invention also provides a pharmaceutical composition comprising a stable Tiotropium bromide solvate having a purity of at least 99.5% area as measured by HPLC, and with less than about 0.15% area by HPLC of 2,2-dithienyl glycolic acid, and pharmaceutically acceptable excipients.

The present invention further provides a process for preparing pharmaceutical composition comprising stable Tiotropium bromide solvate with a purity of at least 99% area by HPLC, and containing less than about 0.15%-area as measured by HPLC of 2,2-dithienyl glycolic acid, and pharmaceutically acceptable excipients. The pure and stable Tiotropium bromide can be micronized to prepare material suitable for formulation. Typically, the term “suitable for formulation” in reference to micronized Tiotropium bromide corresponds to Tiotropium bromide having at least 90% of the particles below 20 microns. The micronization process can, optionally, be followed by a process comprising exposing the micronized form to a suitable solvent to restore the initial content of solvent in the solvate. Usually, the term “suitable solvent” corresponds to the kind of solvent in the original solvated form.

The present invention further provides a method of treating asthma or chromic pulmonary disease by administration of an effective amount of a pharmaceutical composition comprising stable Tiotropium bromide solvate having a purity of at least 99% area as measured by HPLC, and containing less than about 0.15% area as measured by HPLC of 2,2-dithienyl glycolic acid, and pharmaceutical excipients.

The present invention provides the use of the stable Tiotropium bromide solvate with a purity of at least 99% area as measured by HPLC, and containing less than about 0.15% area as measured by HPLC of 2,2-dithienyl glycolic acid, of the present invention for the manufacture of a pharmaceutical composition.

Methods of administration of a pharmaceutical composition of the present invention can be administered in various preparations depending on the age, sex, and symptoms of the patient. The pharmaceutical compositions can be administered, for example, as tablets, pills, powders, liquids, suspensions, emulsions, granules, capsules, suppositories, injection preparations (solutions and suspensions), and the like.

Pharmaceutical compositions of the present invention can optionally be mixed with other forms of Tiotropium bromide solvate and/or other active ingredients such as HMG-CoA reductase inhibitors. In addition, pharmaceutical compositions of the present invention can contain inactive ingredients such as diluents, carriers, fillers, bulking agents, binders, disintegrants, disintegration inhibitors, absorption accelerators, wetting agents, lubricants, glidants, surface active agents, flavoring agents, and the like.

Diluents increase the bulk of a solid pharmaceutical composition and can make a pharmaceutical dosage form containing the composition easier for the patient and care giver to handle. Diluents for solid compositions include, for example, microcrystalline cellulose (e.g., Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, or talc.

Carriers for use in the pharmaceutical compositions may include, but are not limited to, lactose, white sugar, sodium chloride, glucose, urea, starch, calcium carbonate, kaolin, crystalline cellulose, or silicic acid.

Binders help bind the active ingredient and other excipients together after compression. Binders for solid pharmaceutical compositions include for example acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, or starch.

Disintegrants can increase dissolution. Disintegrants include, for example, alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Disintegration inhibitors may include, but are not limited to, white sugar, stearin, coconut butter, hydrogenated oils, and the like Absorption accelerators may include, but are not limited to, quaternary ammonium base, sodium laurylsulfate, and the like.

Wetting agents may include, but are not limited to, glycerin, starch, and the like. Adsorbing agents used include, but are not limited to, starch, lactose, kaolin, bentonite, colloidal silicic acid, and the like.

A lubricant can be added to the composition to reduce adhesion and ease release of the product from a punch or dye during tableting. Lubricants include for example magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Glidants can be added to improve the flowability of non-compacted solid composition and improve the accuracy of dosing. Excipients that can function as glidants include for example colloidal silicon dioxide, magnesium trisilicate, powdered cellulose, starch, talc and tribasic calcium phosphate.

Flavoring agents and flavor enhancers make the dosage form more palatable to the patient. Common flavoring agents and flavor enhancers for pharmaceutical products that can be included in the composition of the present invention include for example maltol, vanillin, ethyl vanillin, menthol, citric acid, fumaric acid, ethyl maltol, and tartaric acid.

Tablets can be further coated with commonly known coating materials such as sugar coated tablets, gelatin film coated tablets, tablets coated with enteric coatings, tablets coated with films, double layered tablets, and multi-layered tablets. Capsules can be coated with shell made, for example, from gelatin and optionally contain a plasticizer such as glycerin and sorbitol, and an opacifying agent or colorant.

Solid and liquid compositions can also be dyed using any pharmaceutically acceptable colorant to improve their appearance and/or facilitate patient identification of the product and unit dosage level.

In liquid pharmaceutical compositions of the present invention, the Tiotropium bromide solvate forms described herein and any other solid ingredients are dissolved or suspended in a liquid carrier, such as water, vegetable oil, alcohol, polyethylene glycol, propylene glycol or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol. Liquid pharmaceutical compositions of the present invention can also contain viscosity enhancing agents to improve the mouth-feel of the product and/or coat the lining of the gastrointestinal tract. Such agents include for example acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar can be added to improve the taste.

Preservatives and chelating agents such as alcohol, sodium benzoate, butylated hydroxy toluene, butylated hydroxyanisole and ethylenediamine tetraacetic acid can be added at safe levels to improve storage stability.

A liquid composition according to the present invention can also contain a buffer such as gluconic acid, lactic acid, citric acid or acetic acid, sodium gluconate, sodium lactate, sodium citrate or sodium acetate.

Selection of excipients and the amounts to use can be readily determined by an experienced formulation scientist in view of standard procedures and reference works known in the art.

A composition for tableting or capsule filing can be prepared by wet granulation. In wet granulation some or all of the active ingredients and excipients in powder form are blended and then further mixed in the presence of a liquid, typically water, which causes the powders to clump up into granules. The granulate is screened and/or milled, dried and then screened and/or milled to the desired particle size. The granulate can then be tableted or other excipients can be added prior to tableting, such as a glidant and/or a lubricant.

A tableting composition can be prepared conventionally by dry blending. For instance, the blended composition of the actives and excipients can be compacted into a slug or a sheet and then comminuted into compacted granules. The compacted granules can be compressed subsequently into a tablet.

As an alternative to dry granulation, a blended composition can be compressed directly into a compacted dosage form using direct compression techniques. Direct compression produces a more uniform tablet without granules. Excipients that are particularly well-suited to direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, only they are not subjected to a final tableting step.

When shaping the pharmaceutical composition into pill form, any commonly known excipient used in the art can be used. For example, carriers include, but are not limited to, lactose, starch, coconut butter, hardened vegetable oils, kaolin, talc, and the like. Binders used include, but are not limited to, gum arabic powder, tragacanth gum powder, gelatin, ethanol, and the like. Disintegrating agents used include, but are not limited to, agar, laminalia, and the like.

For the purpose of shaping the pharmaceutical composition in the form of suppositories, any commonly known excipient used in the art can be used. For example, excipients include, but are not limited to, polyethylene glycols, coconut butter, higher alcohols, esters of higher alcohols, gelatin, semisynthesized glycerides, and the like.

When preparing injectable pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added. If necessary, coloring agents, preservatives, perfumes, seasoning agents, sweetening agents, and other medicines may also be added to the desired preparations during the treatment of schizophrenia.

The amount of Tiotropium bromide solvate or pharmaceutically acceptable salt thereof contained in a pharmaceutical composition for reducing cholesterol according to the present invention is not specifically restricted; however, the dose should be sufficient to treat, ameliorate, or reduce the condition. For example, Tiotropium bromide solvate may be present in an amount of about 1% to about 70%.

The dosage of a pharmaceutical composition for reducing cholesterol according to the present invention will depend on the method of use, the age, sex, weight and condition of the patient. Typically, about 1 mg to 200 mg of Tiotropium bromide solvate may be contained in an administration unit form, preferably a 10 mg tablet.

Having described the invention with reference to certain preferred embodiments, other embodiments will become apparent to one skilled in the art from consideration of the specification. The invention is further defined by reference to the following examples describing in detail the process and compositions of the invention. It will be apparent to those skilled in the art that many modifications, both to materials and methods, may be practiced without departing from the scope of the invention.

### EXAMPLES

| Column: | CPS Hypersil; 5 μm.; 250 × 4.6 mm. | |
|---|---|---|
| Mobile phase: | Eluent A: 3 mL Perchloric acid 70% w/v in | |
| | 1000 mL of water | |
| | Eluent B: Acetonitrile | |
| Gradient | Time (min) | |
| | Mobile Phase B (%) | Mobile Phase A (%) |
| | 0 | 70 |
| | 30 | |
| | 23 | 55 |
| | 45 | |
| | 30 | 50 |
| | 50 | |
| | 35 | 50 |
| | 50 | |
| | 40 | 35 |
| | 65 | |
| | 41 | 70 |
| | 30 | |
| Flow: | 1.8 ml/min | |
| Run time: | 40 min. | |
| Column temperature: | 25° C. | |
| Detector: | UV at 240 nm. | |
| Injection volume: | 5 μl. | |
| Sample preparation: | Tiotropium bromide (1 mg/mL in mobile phase) | |
| Diluent: | Acetonitrile 0.1% V/V; acetic acid in water | |
| | (50:50 V/V) | |
| Post time: | 5 min | |
| In these conditions: | | |
| Retention time | about 3.63 min | |
| tiotropium bromide: | | |
| Retention time | about 5.4 min | |
| dithienylglycolic acid: | | |
| Detection limit: | 0.005%. | |

Stability Tests of Tiotropium Bromide

TABLE 1
Tiotropium Ethanolate dry: stability at 4° C.

| | |
|---|---|
| Time | Dithienylglycolic acid |

| | | |
|---|---|---|
| | | — |
| 11 | days | — |
| 20 | days | — |
| 1 | month | — |
| 2 | months | 0.04% |
| 3 | months | — |

TABLE 2
Tiotropium Ethanolate dry: stability at r.t.

| | |
|---|---|
| Time | Dithienylglycolic acid |

| | | |
|---|---|---|
| | | — |
| 11 | days | — |
| 20 | days | — |
| 1 | month | — |
| 2 | months | 0.04% |
| 3 | month | — |

TABLE 3
Tiotropium monohydrate micronized: stability at 4° C.

| | |
|---|---|
| Time | Dithienylglycolic acid |

| | | |
|---|---|---|
| | | 0.49% |
| 11 | days | 0.49% |
| 20 | days | 0.63% |
| 1 | month | 0.7% |
| 2 | months | 0.82% |
| 3 | months | 0.5% |

TABLE 4
Tiotropium monohydrate micronized: stability at 25° C.

| | |
|---|---|
| Time | Dithienylglycolic acid |

| | | |
|---|---|---|
| | | 0.49% |
| 11 | days | 0.73% |
| 20 | days | 0.58% |
| 1 | month | 0.65% |
| 2 | months | 0.72% |
| 3 | months | 0.58% |

TABLE 5
Tiotropium ethanolate micronized: stability at 4° C.

| Time | Dithienylglycolic acid |
|---|---|
| | — |
| 2 months | — |
| 3 months | — |

### Example 1

### Analysis of the SPIRIVA®HandiHaler®. Capsules

The capsule that were analyzed were part of Lot 408966, expiry date May 2005.

50 mg of sample was dissolved in 50 ml of diluent. The solution was injected, into the chromatographic system equipped with a suitable injection device as blank (as Diluent). The analysis showed that Tiotropium bromide had a purity of 98.94% area by HPLC, and a content of 0.77% of dithienylglycolic acid.

### Example 2

### Preparation of Crude Tiotropium Bromide

0.52 g of N-demethyl tiotropium (1.39 mmol) was suspended in 5.23 mL of CH₃CN under nitrogen.

1.35 g of CH₃Br 50% w/w solution in CH₃CN (0.0071 mol) were loaded, and the suspension was left under stirring at 22° C. for 12 hours. The product was filtered and washed with 1 ml of CH₃CN.

572 mg of wet Tiotropium bromide were obtained (HPLC purity 99.89%, dithienylglycolic acid not detected).

### Example 3

### Preparation of Tiotropium Bromide

4.96 g of N-demethyl tiotropium (13.2 mmol) were loaded in a flask under nitrogen with 49.6 mL of CH₃CN. A suspension was obtained. 12.61 g of CH₃Br 50% w/w —CH₃CN solution- (0.066 mol) were loaded.

The suspension was left under stirring at 22° C. for 64 hours. The product was filtered and washed with 2 mL of CH₃CN.

6.93 g of wet Tiotropium were obtained, and dried under vacuum at 45° C. for 22 h (residual pressure 4 mbar). 5.9 g of dry product (purity 99.8%, dithienylglycolic acid—not detected) were obtained.

### Example 4

### Crystallization of Tiotropium Bromide from Absolute Ethanol

Tiotropium bromide (1.00 g) was dissolved in absolute ethanol (65 ml) at 78° C. The solution was heated to 78° C. for about 30 min, and then was cooled to 22° C. in at least 6 hours. The obtained suspension was maintained at 22° C. for at least 3 hours, and then was filtered on a sintered glass funnel, and the solid was washed two times with absolute ethanol (2×1.0 ml). The solid was dried for 30 min. at 22° C. under N₂ flow, and then for 9 hours at 60° C. under reduced pressure (17 mbar). 0.66 g of Tiotropium bromide (purity 99.68%, dithienylglycolic acid-0.01%) were obtained.

### Example 5

### Crystallization of Tiotropium Bromide from a Mixture of Ethanol and Acetic Acid

65/70° C. until a solution was obtained, and then was cooled to 55° C. in at least 3 hours and at 0±5° C. in at least 3 hours. The obtained suspension was maintained at 0±5° C. for at least 6 hours, and then was filtered on a sintered glass funnel, and the solid was washed two times with ethanol 96%/CH₃COOH 98/2 (3×10.0 ml). The solid was dried for 20 min. at 45° C. under reduced pressure (4 mbar). 16.04 g of Tiotropium bromide was obtained (purity 99.9%, dithienylglycolic acid—not detected).

### Example 6

### Crystallization of Tiotropium Bromide from a Mixture of Ethanol and Acetic Acid

Crude Tiotropium bromide (10 g) is suspended in ethanol 96%/CH₃COOH 98/2 (50 ml). The suspension is heated to 65/70° C. until a solution is obtained, and then it cooled to 55° C. in at least 3 hours and at 0±5° C. in at least 3 hours. The obtained suspension is maintained at 0±5° C. for at least 6 hours, and then it is filtered on a sintered glass funnel, and the solid is washed two times with ethanol 96%/CH₃COOH 98/2 (3×5 ml). The solid is dried for 20 min. at 45° C. under reduced pressure (4 mbar). 8 g of Tiotropium bromide is obtained (purity 99.8%, dithienylglycolic acid 0.13%).

### Example 7

### Crystallization of Tiotropium Bromide from a Mixture of Ethanol and Acetic Acid

Crude Tiotropium bromide (10 g) is suspended in ethanol 96%/CH₃COOH 98/2 (100 ml). The suspension is heated to 65/70° C. until a solution is obtained, and then it is cooled to 55° C. in at least 3 hours and at 0±5° C. in at least 3 hours. The obtained suspension is maintained at 0±5° C. for at least 6 hours, and then it is filtered on a sintered glass funnel, and the solid is washed two times with ethanol 96%/CH₃COOH 98/2 (3×5 ml). The solid is dried for 20 min. at 45° C. under reduced pressure (4 mbar). 8.5 g of Tiotropium bromide is obtained (purity 99.9%, dithienylglycolic acid 0.03%).

### Example 8

### Crystallization of Tiotropium Bromide from a Mixture of Ethanol and Acetic Acid

Crude Tiotropium bromide (10 g) is suspended in ethanol 96%/CH₃COOH 98/2 (200 ml). The suspension is heated to 65/70° C. until a solution is obtained, and then it is cooled to 55° C. in at least 3 hours and at 0±5° C. in at least 3-hours. The obtained suspension is maintained at 0±5° C. for at least 6 hours, and then it is filtered on a sintered glass funnel, and the solid is washed two times with ethanol 96%/CH₃COOH 98/2 (3×5 ml). The solid is dried for 20 min. at 45° C. under reduced pressure (4 mbar). 8.2 g of Tiotropium bromide is obtained (purity 99.9%, dithienylglycolic acid 0.02%).

### Example 9

### Crystallization of Tiotropium Bromide from a Mixture of Methanol and Acetic Acid

Crude Tiotropium bromide (10 g) is suspended in methanol 96%/CH₃COOH 98/2 (200 ml). The suspension is heated to 60/65° C. until a solution is obtained, and then it is cooled to 45° C. in at least 3 hours and at 0±5° C. in at least 3 hours. The obtained suspension is maintained at 0±5° C. for at least 6 hours, and then it is filtered on a sintered glass funnel, and the solid is washed two times with methanol 96%/CH₃COOH 98/2 (3×5 ml). The solid is dried for 20 min. at 45° C. under reduced pressure (4 mbar). 8.2 g of Tiotropium bromide is obtained (purity 99.9%, dithienylglycolic acid 0.02%).

### Example 10

### Crystallization of Tiotropium Bromide from a Mixture of Methanol, Acetone and Acetic Acid

Crude Tiotropium bromide (10 g) is suspended in mixture of methanol/acetone/CH₃COOH 73.5/24.5/2 (50 ml). The suspension is heated to 60/65° C. until a solution is obtained, and then it is cooled to 45° C. in at least 3 hours and at 0±5° C. in at least 3 hours. The obtained suspension is maintained at 0±5° C. for at least 6 hours, and then it is filtered on a sintered glass funnel, and the solid is washed two times with methanol 96%/CH₃COOH 98/2 (3×5 ml). The solid is dried for 20 min. at 45° C. under reduced pressure (4 mbar). 8.2 g of Tiotropium bromide is obtained (purity 99.9%, dithienylglycolic acid 0.05%).

### Example 11

### Crystallization of Tiotropium Bromide from a Mixture of Methanol, Acetone and Acetic Acid

Crude Tiotropium bromide (10 g) is suspended in mixture of methanol/acetone/CH₃COOH 24.5/73.5/2 (50 ml). The suspension is heated to 60/65° C. until a solution is obtained, and then it is cooled to 45° C. in at least 3 hours and at 0±5° C. in at least 3 hours. The obtained suspension is maintained at 0±5° C. for at least 6 hours, and then it is filtered on a sintered glass funnel, and the solid is washed two times with methanol 96%/CH₃COOH 98/2 (3×5 ml). The solid is dried for 20 min. at 45° C. under reduced pressure. 8.2 g of Tiotropium bromide is obtained (purity 99.9%, dithienylglycolic acid 0.05%).

### Example 12

### Crystallization of Tiotropium Bromide from a Mixture of Methanol, Acetone and Acetic Acid

Crude Tiotropium bromide (10 g) is suspended in mixture of methanol/acetone/CH₃COOH 24.5/73.5/2 (100 ml). The suspension is heated to 60/65° C. until a solution is obtained, and then it is cooled to 45° C. in at least 3 hours and at 0±5° C. in at least 3 hours. The obtained suspension is maintained at 0±5° C. for at least 6 hours, and then it is filtered on a sintered glass funnel, and the solid is washed two times with methanol 96%/CH₃COOH 98/2 (3×5 ml). The solid is dried for 20 min. at 45° C. under reduced pressure. 7.7 g of Tiotropium bromide is obtained (purity 99.9%, dithienylglycolic acid 0.03%).

### Example 13

### Crystallization of Tiotropium Bromide from a Mixture of Methanol, Acetone and Acetic Acid

Crude Tiotropium bromide (10 g) is suspended in mixture of methanol/acetone/CH₃COOH 24.5/73.5/2 (300 ml). The suspension is heated to 60/65° C. until a solution is obtained, and then it is cooled to 45° C. in at least 3 hours and at 0±5° C. in at least 3 hours. The obtained suspension is maintained at 0±5° C. for at least 6 hours, and then it is filtered on a sintered glass funnel, and the solid is washed two times with methanol 96%/CH₃COOH 98/2 (3×5 ml). The solid is dried for 20 min. at 45° C. under reduced pressure. 5.1 g of Tiotropium bromide is obtained (purity 99.9%, dithienylglycolic acid not detectable).

### Example 14

### Crystallization of Tiotropium Bromide from a Mixture of N-Propanol, Water and Acetic Acid

Crude Tiotropium bromide (10 g) is suspended in n-propanol 93/water 5/CH₃COOH 2 (200 ml). The suspension is heated to 60/65° C. until a solution is obtained, and then it is cooled to 45° C. in at least 3 hours and at 0±5° C. in at least 3 hours. The obtained suspension is maintained at 0±5° C. for at least 6 hours, and then it is filtered on a sintered glass funnel, and the solid is washed two times with n-propanol 96%/CH₃COOH 98/2 (3×5 ml). The solid is dried for 20 min. at 45° C. under reduced pressure. 8.2 g of Tiotropium bromide is obtained (purity 99.9%, dithienylglycolic acid 0.02%).

### Example 15

### Crystallization of Tiotropium Bromide from a Mixture of N-Propanol and Acetic Acid

Wet crude Tiotropium bromide (10 g) is suspended in n-propanol 98/CH₃COOH 2 (8500 ml). The suspension is heated to 60/65° C. until a solution is obtained, and then it is cooled to 45° C. in at least 3 hours and at 0±5° C. in at least 3 hours. The obtained suspension is maintained at 0±5° C. for at least 6 hours, and then it is filtered on a sintered glass funnel, and the solid is washed two times with n-propanol 96%/CH₃COOH 98/2 (3×5 ml). The solid is dried for 20 min. at 45° C. under reduced pressure. 4.2 g of Tiotropium bromide is obtained (purity 99.9%, dithienylglycolic acid not detectable).

### Example 16

### Crystallization of Tiotropium Bromide from a Mixture of N-Propanol and Acetic Acid

Wet crude Tiotropium bromide (10 g) is suspended in n-propanol 98/CH₃COOH 2 (50 ml). The suspension is heated to 60/65° C. until a solution is obtained, and then it is cooled to 45° C. in at least 3 hours and at 0±5° C. in at least 3 hours. The obtained suspension is maintained at 0±5° C. for at least 6 hours, and then it is filtered on a sintered glass funnel, and the solid is washed two times with n-propanol 96%/CH₃COOH 98/2 (3×5 ml). The solid is dried for 20 min. at 45° C. under reduced pressure. 8.2 g of Tiotropium bromide is obtained (purity 99.9%, dithienylglycolic acid 0.08).

### Example 17

### Crystallization of Tiotropium Bromide from a Mixture of N-Propanol and Acetic Acid

Wet crude Tiotropium bromide (10 g), is suspended in n-propanol 98/CH₃COOH 2 (100 ml). The suspension is heated to 60/65° C. until a solution is obtained, and then it is cooled to 45° C. in at least 3 hours and at 0±5° C. in at least 3 hours. The obtained suspension is maintained at 0±5° C. for at least 6 hours, and then it is filtered on a sintered glass funnel, and the solid is washed two times with n-propanol 96%/CH₃COOH 98/2 (3×5 ml). The solid is dried for 20 min. at 45° C. under reduced pressure. 4.2 g of Tiotropium bromide is obtained (purity 99.9%, dithienylglycolic acid 0.03).

### Example 18

### Preparation of Tiotropium Bromide from AcOH/MeOH/n-Heptan 7/2/2.3

Tiotropium bromide (1.00 g) was dissolved at 45° C. with a mixture 7/2 (V/V) of acetic acid/methanol (11 ml), the solution was heated to 45° C. for 1.5 hours and n-heptane (2.75 ml) was then added drop-wise in at least 20 min. The obtained solution was heated to 45° C. for one hour (no solid formation observed), was cooled to 23.5° C. in at least 3 hours and the suspension was maintained at 23.5° C. for at least 3 hours. After filtration on a sintered glass funnel, the solid was washed three times with 3.0 mL of n-heptane. Drying for 16 hours at 60° C. under reduced pressure (18 mbar), yielded 0.67 g of Tiotropium bromide (purity 99.9%, dithienylglycolic acid 0.03).

### Example 19

### Micronization Process

Tiotropium Bromide was micronized to obtain P.S.D target of:
Min. 80%<5.84 μm
Min. 70% between 0.6 and 10 microns
The micronizer in use was a Jet-mill MC 50 (made by Micro-Macinazionne). 32°05′ angle nozzles were installed.
Nitrogen was used as the micronization gas.
Micronization air Pressure was 10 bars.
Feed rate was 0.2 kg/hr.
The micronized Tiotropium bromide obtained by the above process has a PSD value:
80%≦5.84 μm
93.76% between 0.6 and 10 microns.

What is claimed:

1. Tiotropium bromide solvate of the following formula: 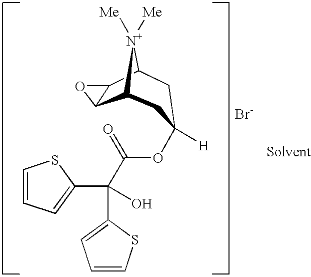
having a purity of at least 99%.

2. The Tiotropium bromide solvate of claim 1, wherein the Tiotropium bromide has a purity ranging from about 99% to about 100%.

3. The Tiotropium bromide solvate of claim 2, wherein the Tiotropium bromide has a purity ranging from about 99.5% to about 100%.

4. The Tiotropium bromide of solvate claim 3, wherein the Tiotropium bromide has a purity ranging from about 99.7% to about 100%.

5. The Tiotropium bromide solvate of claims 1, 2, 3, or 4, wherein the Tiotropium bromide includes less than about 0.15% of 2,2-dithienyl glycolic acid.

6. A Tiotropium bromide solvate with less than about 0.15% of 2,2-dithienyl glycolic acid.

7. The Tiotropium bromide solvate of claim 6, wherein the Tiotropium bromide contains from about 0.15% to the detection limit of an HPLC method of 2,2-dithienyl glycolic acid.

8. The Tiotropium bromide solvate of claim 7, wherein the Tiotropium bromide contains less than about 0.05% of 2,2-dithienyl glycolic acid.

9. The Tiotropium bromide solvate of claim 8, wherein the Tiotropium bromide contains from about 0.05% to the detection limit of an HPLC method of 2,2-dithienyl glycolic acid.

10. The Tiotropium bromide solvate of claim 6, wherein the Tiotropium bromide contains less than about 0.02% of 2,2-dithienyl glycolic acid.

11. The Tiotropium bromide solvate of claim 10, wherein the Tiotropium bromide contains from about 0.02% to the detection limit of an HPLC method of 2,2-dithienyl glycolic acid.

12. The Tiotropium bromide solvate of claim 6, wherein the Tiotropium bromide contains less than about 0.01% of 2,2-dithienyl glycolic acid.

13. The Tiotropium bromide solvate of claim 12, wherein the Tiotropium bromide contains from about 0.01% to the detection limit of an HPLC method of 2,2-dithienyl glycolic acid.

14. A stable Tiotropium bromide solvate.

15. The stable Tiotropium bromide solvate of claim 14, wherein the level of 2,2-dithienyl glycolic acid does not increase to more than 0.15%, when maintained at a temperature ranging from about 4° C. to about 30° C., for at least about two months.

16. The Tiotropium bromide solvate of claims 1 or 6, wherein the Tiotropium bromide solvate is stable.

17. The Tiotropium bromide solvate of any of the claims 1, 6 or 14, wherein the Tiotropium bromide solvate is selected from the group consisting of an acetic acid solvate and an alcohol solvate.

18. The solvate of claim 17, wherein the alcohol solvate is a C₁₋₈ alcoholate.

19. The solvate of claim 18, wherein the C₁₋₈ alcoholate is C₁₋₆ alcoholate.

20. The solvate of claim 19, wherein the C₁₋₆ alcoholate is C₁₋₅ alcoholate.

21. The solvate of claim 20, wherein the C₁₋₅ alcoholate is C₁₋₄ alcoholate.

22. The solvate of claim 21, wherein the C₁₋₄ alcoholate is selected from the group consisting of methanolate, ethanolate, isopropanolate, n-propanolate and n-butanolate.

23. The solvate of claim 22, wherein the C₁₋₄ alcoholate is selected from the group consisting of methanolate, ethanolate or n-propanolate.

24. An HPLC method comprising:
(a) combining a Tiotropium bromide sample with a mixture of acetonitrile:acetic acid in water in a ratio of about 0.1%:99.9%, to obtain a solution;
(b) injecting the solution into a column;
(c) eluting the sample from the column at about 3.63 min using an eluent mixture comprised of a first eluent and a second eluent, the first eluent comprising perchloric acid:water in a ratio of 7:3 and the second eluent comprising acetonitrile; and
(d) measuring the 2,2-dithienyl glycolic acid content in the relevant sample with a UV detector.

25. The HPLC method of claim 24, wherein said column is a 250×4.6 mm×0.5 μm CPS Hypersil (or similar) column.

26. The method of claim 24, wherein the ratio of the first and second eluents varies over time.

27. The method of claim 26, wherein at the time 0 minutes, the eluent contains 70% of the first eluent and 300% of the second eluent.

28. The method of claim 26, wherein at the time 23 minutes, the eluent contains 55% of the first eluent and 45% of the second eluent.

29. The method of claim 26, wherein at the time 30 minutes, the eluent contains 50% of the first eluent and 50% of the second eluent.

30. The method of claim 26, wherein at the time 35 minutes, the eluent contains 50% of the first eluent and 50% of the second eluent.

31. The method of claim 26, wherein at the time 40 minutes, the eluent contains 35% of the first eluent and 65% of the second eluent.

32. The method of claim 26, wherein at the time 41 minutes, the eluent contains 70% of the first eluent and 30% of the second eluent.

33. The method of claim 24, wherein the 2,2-dithienyl glycolic acid content is measured at a wave length of 240 nm.

34. A process for preparing stable Tiotropium bromide solvate having a purity of at least 99% and containing less than about 0.15% of 2,2-dithienyl glycolic acid, comprising crystallizing Tiotropium bromide from a solvent system comprising an organic acid, wherein the ratio of Tiotropium bromide to the solvent system is at least about 1 to about 5.

35. A process for preparing stable Tiotropium bromide solvate having a purity of at least 99% and with less than about 0.05% of 2,2-dithienyl glycolic acid as measured by HPLC, comprising crystallizing Tiotropium bromide from a solvent system comprising an organic acid, wherein the ratio of Tiotropium bromide to the solvent system is at least about 1 to about 10.

36. A process for preparing stable Tiotropium bromide solvate having a purity of at least 99% and with less than about 0.02% of 2,2-dithienyl glycolic acid, comprising crystallizing Tiotropium bromide from a solvent system comprising an organic acid, wherein the ratio of Tiotropium bromide to the solvent system is of at least about 1 to about 20.

37. A process for preparing stable Tiotropium bromide solvate having a purity of at least 99% and with less than about 0.01% of 2,2-dithienyl glycolic acid, comprising comprising an organic acid, wherein the ratio of Tiotropium bromide to the solvent system is of at least about 1 to about 30.

38. The process of claims 34, 35, 36, or 37, wherein the organic acid is selected from the group consisting of trifluoroacetic acid, tartaric acid, maleic acid, propionic acid, oxalic acid, p-toluen sulphonic acid, methan sulphonic acid and acetic acid.

39. The process of claim 38, wherein the organic acid is acetic acid.

40. The processes of claims 34, 35, 36, or 37, further comprising cooling the solution to obtain a suspension.

41. The process of claim 40, wherein the solvent system further comprises an alcohol.

42. The process of claim 41, wherein the alcohol is a C₁₋₈ alcohol.

43. The process of claim 42, wherein the C₁₋₈ alcohol is C₁₋₆ alcohol.

44. The process of claim 43, wherein the C₁₋₆ alcohol is C₁₋₅ alcohol.

45. The process of claim 44, wherein the C₁₋₅ alcohol is C₁₋₄ alcohol.

46. The process of claim 45, wherein the C₁₋₄ alcohol is selected from the group consisting of methanol, ethanol, isopropanol, n-propanol and n-butanol.

47. The process of claim 46, wherein the C₁₋₄ alcoholate is selected from the group consisting of methanol, ethanol and n-propanol.

48. The process of claims 34, 35, 36, or 37, further comprising heating the combination of Tiotropium bromide and the solvent system.

49. The process of claim 48, wherein the heating is to a temperature ranging from about 60° C. to about 78° C.

50. The process of claim 40, wherein the solution is cooled to a temperature ranging from about 25° C. to about 0° C.

51. The process of claim 50, wherein the cooling is done over a period of about 4 to about 10 hours.

52. The process of claim 40, wherein the process further comprises recovering the crystallized product.

53. A pharmaceutical composition comprising a stable. Tiotropium bromide solvate having a purity of at least 99%, and with less than about 0.15% of 2,2-dithienyl glycolic acid, and pharmaceutically acceptable excipients.

54. A process for preparing pharmaceutical composition comprising stable Tiotropium bromide solvate with a purity of at least 99%, and with less than about 0.15% of 2,2-dithienyl glycolic acid, and pharmaceutically acceptable excipients.

55. A method of treating asthma or chromic pulmonary disease by administration of an effective amount of a pharmaceutical composition comprising stable Tiotropium bromide solvate having a purity of at least 99%, and with less than about 0.15% of 2,2-dithienyl glycolic acid, and pharmaceutical excipients.

56. The use of the stable Tiotropium bromide solvate with a purity of at least 99%, and with less than about 0.15% of 2,2-dithienyl glycolic acid, of the present invention for the manufacture of a pharmaceutical composition.

57. The Triotropium bromide solvate of claims 1, 6, or 14, wherein the tiotropium bromide solvate is micronized.

58. A process for preparing Tiotropium bromide including less than about 0.15% area by HPLC of 2,2-dithienyl glycolic acid comprising the steps of:
(a) obtaining one or more samples of one or more Tiotrpium bromide batches;
(b) measuring a level of 2,2-dithienyl glycolic acid in each of the samples by HPLC;
(c) selecting the Tiotropium bromide batch that comprises a level of 2,2-dithienyl glycolic acid of less than about 0.15% area by HPLC; and
(d) using the selected batch to prepare a Tiotropium bromide comprising less than about: 0.15% area by HPLC of 2,2-dithienyl glycolic acid.

59. The process of claim 58, further comprising purifying the samples prior to measurement.
